(19) United States
(12) Patent Application Publication
(10) Pub. No.: US 2004/0258627 A1
(43) Pub. Date: Dec. 23, 2004

(54) Self-foaming, foam-like, after-foaming or foamable cosmetic or dermatological preparation

(75) Inventors: Heidi Riedel, Hamburg (DE); Jens Schulz, Schenefeld (DE); Anja Goppel, Hamburg (DE); Andreas Bleckmann, Ahrensburg (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/843,552

(22) Filed: May 10, 2004

Related U.S. Application Data
(63) Continuation of application No. PCT/EP02/12350, filed on Nov. 6, 2002.

(30) Foreign Application Priority Data
Nov. 9, 2001 (DE) 101 55 956.9

(51) Int. Cl. A61K 9/00

(52) U.S. Cl. 424/47

(57) ABSTRACT

The present invention is a self-foaming, foamed, post-foaming or foamable cosmetic and dermatological preparation comprising I) an emulsifier system comprising A) at least one emulsifier selected from the group of fully-, partially- and non-neutralized, branched and unbranched, saturated and unsaturated fatty acids having a chain length of 1-40 carbon atoms, B) at least one emulsifier selected from the group of polyethoxylated fatty acid esters having a chain length of 10 to 40 carbon atoms and a degree of ethoxylation of 5 to 100, and C) at least one coemusifier selected from the group of saturated and unsaturated, branched and unbranched fatty alcohols having a chain length of 10 to 40 carbon atoms, and II) up to 30% by weight—based on the total weight of the preparation—of a lipid phase comprising a) one or several lipids selected from the group consisting of silicone oils and silicon waxes and b) one or several lipids from the group of nonpolar lipids having a polarity ≧30 mN/m, the ratio of (a) to (b) ranging from 1:3-3:1. The present invention also includes a method of preparing foam comprising combining the cosmetic or dermatological preparation with at least one filler material gas or propellant are releasing the preparation and the filler material gas or propellant from a pressurized gas container.

### CROSS-REFERENCE TO RELATED APPLICATIONS

This is a continuation application of PCT/EP02/12350, filed Nov. 6, 2002, which is incorporated herein by reference in its entirety, and also claims the benefit of German Priority Application No. 101 55 956.9, filed Nov. 9, 2001.

### FIELD OF THE INVENTION

The present invention relates to self-foaming, foam-like, after-foaming or foamable cosmetic and dermatological preparations, in particular skincare cosmetic and dermatological preparations.

### BACKGROUND OF THE INVENTION

Foams or foam-like preparations are a type of disperse system.

By far the most important and best known disperse system are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semi-liquid, high-viscosity or solid cell ribs. The cell ribs, connected via points of intersection, form a continuous framework. The foam lamellae stretch between the cell ribs (closed-cell foam). If the foam lamellae are disturbed or if they flow back into the cell rib at the end of foam formation, an open-cell foam is obtained. Foams are also thermodynamically unstable since a reduction in the surface area leads to the production of surface energy. The stability and thus the existence of a foam is thus dependent on to what extent it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of hair care.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A disadvantage of the prior art is that such foams have only low stability, for which reason they usually collapse within approximately 24 hours. A requirement of cosmetic preparations, however, is that they have stability for years, as far as possible. This problem is generally taken into account by the fact that the consumer produces the actual foam himself just before use using a suitable spray system, for which purpose, for example, it is possible to use spray cans in which a liquefied pressurized gas serves as propellant gas. Upon opening the pressure valve, the propellant liquid mixture escapes through a fine nozzle, and the propellant evaporates, leaving behind a foam.

After-foaming cosmetic preparations are also known per se. They are firstly applied to the skin from an aerosol container in flowable form and, after a short delay, develop the actual foam only once they are on the skin under the effect of the after-foaming agent present, for example a shaving foam. After-foaming preparations are often in specific formulation forms, such as, for example, after-foaming shaving gels or the like.

However, the prior art does not include any sort of cosmetic or dermatological preparations which could be foamed as early as during the preparation and nevertheless have a sufficiently high stability in order to be packaged in the usual manner, stored and put onto the market.

### SUMMARY OF THE INVENTION

An object of the present invention was therefore to enrich the prior art and to provide cosmetic or dermatological self-foaming or foam-like preparations which do not have the disadvantages of the prior art.

German laid-open specification DE 197 54 659 discloses that carbon dioxide is a suitable active ingredient for stabilizing or increasing the epidermal ceramide synthesis rate, which may serve to enhance the permeability barrier, reduce the transepidermal water loss and increase the relative skin moisture. To treat the skin, the CO₂ is, for example, dissolved in water, which is then used to rinse the skin. However, the prior art hitherto does not include any sort of cosmetic or dermatological bases in which a gaseous active ingredient could be incorporated in an adequate, i.e. effective, concentration.

It was thus a further object of the present invention to find cosmetic or dermatological bases into which effective amounts of gaseous active ingredients can be incorporated.

It was surprising and could not have been foreseen by the person skilled in the art that self-foaming, foam-forming, after-foaming or foamable cosmetic and dermatological preparations which consist of:

I. an emulsifier system which consists of

A. at least one emulsifier A chosen from the group of completely neutralized, partially neutralized or unneutralized branched or unbranched, saturated or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C. at least one coemulsifier C chosen from the group of saturated or unsaturated, branched or unbranched fatty alcohols with a chain length of from 10 to 40 carbon atoms, and

II. up to 30% by weight—based on the total weight of the preparation—of a lipid phase which comprises

(a) one or more lipids from the group of silicone oils and silicone waxes and

(b) one or more lipids from the group of nonpolar lipids with a polarity of ≧30 mN/m,

where the ratio of (a) to (b) is chosen from the range 1:3 to 3:1,

overcome the disadvantages of the prior art.

### DETAILED DESCRIPTION OF THE PREFERRED EMBODIMENTS

For the purposes of the present invention, “self-foaming”, “foam-like”, “after-foaming” and “foamable” are understood as meaning preparations from which foams can in principle be produced by introducing one or more gases—whether during the production process, or during use by the consumer or in another way. In such foams, the gas bubbles are present in (any) distributed form in one (or more) liquid phase(s), the (foamed) preparations do not necessarily having to have the appearance of a foam in macroscopic terms. (Foamed) cosmetic or dermatological preparations according to the invention (for the sake of simplicity referred to below as foams) may, for example, be macroscopically visibly dispersed systems of gases dispersed in liquids. The foam character can, however, for example, be visible only under a (light) microscope. Moreover, foams according to the invention are—particularly when the gas bubbles are too small to be recognized under a light microscope—also recognizable from the sharp increase in the volume of the system.

According to the prior art, self-foaming, foam-like, after-foaming or foamable cosmetic emulsions cannot be formulated or prepared industrially without the use of particular surfactants. This is true particularly for systems which are based on classic emulsifiers. According to the prior art, systems of this type, with the addition of propellant gas, developed exclusively aqueous-moist foams which rapidly broke following application.

As a result of the invention, a rich, compact cream foam is obtainable for the first time which is characterized by a long storage period, and by an extraordinarily high stability and a compact appearance.

The preparations according to the invention are extremely satisfactory preparations in every respect. It was particularly surprising that the foams obtainable from the preparations according to the invention—even in cases of an unusually high gas volume—are extraordinarily stable. Accordingly, they are particularly suitable for use as bases for preparation forms having diverse intended uses. The preparations according to the invention have very good sensory properties such as, for example, extensibility on the skin or the ability to be absorbed into the skin, and, moreover, are characterized by above-average skin care.

According to the invention, fine-bubbled, rich foams of excellent cosmetic elegance are obtainable. In addition, preparations which are particularly readily tolerated by the skin are obtainable, where it is possible for valuable ingredients to be distributed on the skin particularly readily.

The invention further provides for the use of self-foaming, foam-like, after-foaming or foamable cosmetic or dermatological preparations which

I. an emulsifier system which consists of

A. at least one emulsifier A chosen from the group of completely neutralized, partially neutralized or unneutralized branched or unbranched, saturated or unsaturated fatty acids with a chain length of from 10 to 40 carbon atoms,

B. at least one emulsifier B chosen from the group of polyethoxylated fatty acid esters with a chain length of from 10 to 40 carbon atoms and with a degree of ethoxylation of from 5 to 100 and

C. at least one coemulsifier C chosen from the group of saturated or unsaturated, branched or unbranched fatty alcohols with a chain length of from 10 to 40 carbon atoms, and

II. up to 30% by weight—based on the total weight of the preparation—of a lipid phase which comprises

(a) one or more lipids from the group of silicone oils and silicone waxes and

(b) one or more lipids from the group of nonpolar lipids with a polarity of ≧30 mN/m,

where the ratio of (a) to (b) is chosen from the range 1:3 to 3:1,

as cosmetic or dermatological bases for gaseous active ingredients.

The emulsifier(s) A is/are preferably chosen from the group of fatty acids which have been completely or partially neutralized with customary alkalis (such as, for example, sodium hydroxide or potassium hydroxide, sodium carbonate or potassium carbonate, and mono- or triethanolamine). Stearic acid and stearates, isostearic acid and isostearates, palmitic acid and palmitates, and myristic acid and myristates, for example, are particularly advantageous.

The emulsifier(s) B is/are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polylethoxylated stearic esters.

The coemulsifier(s) C is/are preferably chosen according to the invention from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols, which are the unsaponifiable alcohol fraction of wool wax which is obtained following the saponification of wool wax). Particular preference is given to cetyl alcohol and cetylstearyl alcohol.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. Particular preference is given to a weight ratio of approximately 1:1:1.

It is advantageous for the purposes of the present invention to choose the total amount of emulsifiers A and B and of coemulsifier C from the range from 2 to 20% by weight, advantageously from 5 to 15% by weight, in particular from 7 to 13% by weight, in each case based on the total weight of the formulation.

For the purposes of the present invention, it is particularly preferred if the gas phase of the preparations comprises carbon dioxide or consists entirely of carbon dioxide. It is particularly advantageous if carbon dioxide is a or the active ingredient in the preparations according to the invention.

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

Preferably, for the purposes of the present invention, the further emulsifier(s) is/are chosen from the group of hydrophilic emulsifiers. According to the invention, particular preference is given to mono-, di- and tri-fatty acid esters of sorbitan.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate or glyceryl laurate.

Particularly advantageous nonpolar lipids for the purposes of the present invention are those listed below.

| | | | Polarity |
|---|---|---|---|
| Manufacturer | Trade name | INCI name | mN/m |
| Total SA | Ecolane 130 | Cycloparaffin | 49.1 |
| Neste PAO N.V. | Nexbase 2006 FG | Polydecene | 46.7 |
| (Supplier Hansen & Rosenthal) | | | |
| Chemische Fabrik Lehrte | Polysynlane | Hydrogenated | 44.7 |
| | | Polyisobutene | |
| EC Erdölchemie (Supplier Bayer AG) | Solvent ICH | Isohexadecane | 43.8 |
| DEA Mineral oil (Supplier | Pionier 2076 | Mineral Oil | 43.7 |
| Hansen & Rosenthal) | | | |
| Tudapetrol | | | |
| DEA Mineral oil (Supplier | Pionier 6301 | Mineral Oil | 43.7 |
| Hansen & Rosenthal) | | | |
| Tudapetrol | | | |
| EC Erdölchemie GmbH | Isoeicosane | Isoeicosane | 41.9 |
| Condea Chemie | Isofol 1212 | | 40.3 |
| | Carbonate | | |
| Gattefossé | Softcutol O | Ethoxydiglycol Oleate | 40.5 |
| Creaderm | Lipodermanol OL | Decyl Olivate | 40.3 |
| Henkel | Cetiol S | Dioctylcyclohexane | 39.0 |
| DEA Mineral oil (Supplier | Pionier 2071 | Mineral Oil | 38.3 |
| Hansen & Rosenthal) | | | |
| Tudapetrol | | | |
| WITCO BV | Hydrobrite 1000 | Paraffinum Liquidum | 37.6 |
| | PO | | |
| Goldschmidt | Tegosoft HP | Isocetyl Palmitate | 36.2 |
| Condea Chemie | Isofol Ester 1693 | | 33.5 |
| Condea Chemie | Isofol Ester 1260 | | 33.0 |
| Unichema | Prisorine 2036 | Octyl Isostearate | 31.6 |
| Henkel Cognis | Cetiol CC | Dicaprylyl Carbonate | 31.7 |
| ALZO (ROVI) | Dermol 99 | Trimethylhexyl | 31.1 |
| | | Isononanoate | |
| ALZO (ROVI) | Dermol 89 | 2-Ethylhexyl | 31.0 |
| | | Isononanoate | |
| Unichema | Estol 1540 EHC | Octyl Cocoate | 30.0 |

Of the hydrocarbons, paraffin oil, and further hydrogenated polyolefins, such as hydrogenated polyisobutenes, squalane and squalene, in particular, are to be used advantageously for the purposes of the present invention.

For the purposes of the present invention, advantageous silicone oils and silicone waxes are cyclic or linear silicone oils and silicone waxes.

Silicone oils are high molecular weight synthetic polymeric compounds in which silicon atoms are linked in a chain-like or network-like manner via oxygen atoms, and the remaining valences of the silicon are saturated by hydrocarbon radicals (in most cases methyl, less often ethyl, propyl, phenyl groups etc.). Systematically, the silicone oils are referred to as polyorganosiloxanes; the methyl-substituted polyorganosiloxanes, which represent the most significant compounds of this group in terms of number and are characterized by the following structural formula

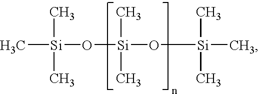

are also referred to as polydimethylsiloxane or Dimethicone (INCI). There are dimethicones with various chain lengths and with various molecular weights.

Dimethicones with different chain lengths and phenyltrimethicones are particularly advantageous linear silicone oils for the purposes of the present invention.

Particularly advantageous polyorganosiloxanes for the purposes of the present invention are also, for example, dimethylpolysiloxanes [poly(dimethylsiloxane)], which are available, for example, under the trade names Abil 10 to 10 000 from Th. Goldschmidt. Also advantageous are phenylmethylpolysiloxanes (INCI: Phenyl Dimethicone, Phenyl Trimethicone), cyclic silicones (octamethylcyclotetrasiloxane or decamethylcyclopentasiloxane), which are also referred to as Cyclomethicones in accordance with INCI, amino-modified silicones (INCI: Amodimethicones) and silicone waxes, e.g. polysiloxane-polyalkylene copolymers (INCI: Stearyl Dimethicone and cetyl Dimethicone) and dialkoxydimethylpolysiloxanes (Stearoxy Dimethicone and Behenoxy Stearyl Dimethicone), which are available as various Abil wax grades from Th. Goldschmidt.

Particularly advantageous cyclic silicone oils for the purposes of the present invention are cyclomethicones, in particular cyclomethicones D5 or cyclomethicones D6.

Cyclic silicones to be used advantageously according to the invention are generally characterized by structural elements as follows

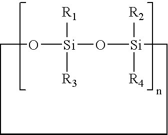

where the silicon atoms may be substituted by identical or different alkyl radicals or aryl radicals, which are shown here in general terms by the radicals R₁-R₄ (that is to say the number of different radicals is not necessarily limited to 4). n can assume values from 3/2 to 20. Fractional values for n take into consideration that uneven numbers of siloxy groups may be present in the cycle.

Also particularly advantageous for the purposes of the present invention are the silicone oils listed below:

| Manu- | | | Polarity |
|---|---|---|---|
| facturer | Trade name | INCI name | mN/m |
| Wacker | Wacker Silicone Oil AK | Polydimethylsiloxane | 26.9 |
| | 100 | | |
| Wacker | Wacker Silicone Oil AK | Polydimethylsiloxane | 46.5 |
| | 50 | | |
| Wacker | Wacker Silicone Oil AK | Polydimethylsiloxane | 42.4 |
| | 35 | | |
| Wacker | Wacker Silicone Oil AK | Polydimethylsiloxane | 40.9 |
| | 20 | | |
| Dow | Dow Corning Fluid 245 | Cyclopentasiloxane | 32.3 |
| Corning | | | |
| Dow | Dow Corning Fluid 345 | Cyclomethicone | 28.5 |
| Corning | | | |

It is particularly advantageous for the purposes of the present invention to choose the ratio of nonpolar lipids to silicone oils to be about 1:1.

The content of the lipid phase is advantageously chosen to be less than 30% by weight, preferably between 2.5 and 30% by weight, particularly preferably between 5 and 15% by weight, in each case based on the total weight of the preparation. It may also be advantageous, although it is not obligatory, for the lipid phase to comprise up to 40% by weight, based on the total weight of the lipid phase, of polar lipids (having a polarity of ≦20 mN/m) or medium-polarity lipids (having a polarity of from 20 to 30 mN/m).

For the purposes of the present invention, particularly advantageous polar lipids are all native lipids, such as, for example, olive oil, sunflower oil, soybean oil, peanut oil, rapeseed oil, almond oil, palm oil, coconut oil, castor oil, wheatgerm oil, grapeseed oil, thistle oil, evening primrose oil, macadamia nut oil, corn oil, avocado oil and the like and those listed below.

| | | | Polarity |
|---|---|---|---|
| Manufacturer | Trade name | INCI name | mN/m |
| Condea Chemie | Isofol 14 T | Butyl Decanol (+) Hexyl Octanol (+) | 19.8 |
| | | Hexyl Decanol (+) Butyl Octanol | |
| Lipochemicals | Lipovol MOS-130 | Tridecyl Stearate(+) Tridecyl | 19.4 |
| INC./USA | | Trimellitate(+) Dipentaerythrityl | |
| (Induchem) | | Hexacaprylate/Hexacaprate | |
| | Castor oil | | 19.2 |
| CONDEA Chemie | Isofol Ester 0604 | | 19.1 |
| Huels | Miglyol 840 | Propylene Glycol | 18.7 |
| CONDEA Chemie | | Dicaprylate/Dicaprate | |
| CONDEA Chemie | Isofol 12 | Butyl Octanol | 17.4 |
| Goldschmidt | Tegosoft SH | Stearyl Heptanoate | 17.8 |
| | Avocado oil | | 14.5 |
| Henkel Cognis | Cetiol B | Dibutyl Adipate | 14.3 |
| ALZO (ROVI) | Dermol 488 | PEG 2 Diethylene Hexanoate | 10.1 |
| Condea Augusta | Cosmacol ELI | C₁₂₋₁₃ Alkyl Lactate | 8.8 |
| S.P.A. | | | |
| ALZO (ROVI) | Dermol 489 | Diethylene Glycol Dioctanoate(/ | 8.6 |
| | | Diisononanoate | |
| Condea Augusta | Cosmacol ETI | Di-C_{12/13} Alkyl Tartrate | 7.1 |
| S.P.A. | | | |
| Henkel Cognis | Emerest 2384 | Propylene Glycol | 6.2 |
| | | Monoisostearate | |
| Henkel Cognis | Myritol 331 | Cocoglycerides | 5.1 |
| Unichema | Prisorine 2041 | Triisostearin | 2.4 |
| | GTIS | | |

Particularly advantageous medium-polar lipids for the purposes of the present invention are those listed below

| | | | Polarity |
|---|---|---|---|
| | | | (Water) |
| Manufacturer | Trade name | INCI name | mN/m |
| Henkel Cognis | Cetiol OE | Dicaprylyl Ether | 30.9 |
| | Dihexyl carbonate | Dihexyl Carbonate | 30.9 |
| Albemarle S.A. | Silkflo 366 NF | Polydecene | 30.1 |
| Stearinerie Dubois Fils | DUB VCI 10 | Isodecyl Neopentanoate | 29.9 |
| ALZO (ROVI) | Dermol IHD | Isohexyl Decanoate | 29.7 |
| ALZO (ROVI) | Dermol 108 | Isodecyl Octanoate | 29.6 |
| | Dihexyl Ether | Dihexyl Ether | 29.2 |
| ALZO (ROVI) | Dermol 109 | Isodecyl 3,5,5 Trimethyl | 29.1 |
| | | Hexanoate | |
| Henkel Cognis | Cetiol SN | Cetearyl Isononanoate | 28.6 |
| Unichema | Isopropyl | Isopropyl Palmitate | 28.8 |
| | palmitate | | |
| Dow Corning | DC Fluid 345 | Cyclomethicone | 28.5 |
| Dow Corning | Dow Corning Fluid | Cyclopolydimethylsiloxane | 28.5 |
| | 244 | | |
| Nikko Chemicals | Jojoba oil Gold | | 26.2 |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic Acid 3,5,5 | 26.2 |
| | | Trimethyl Ester | |
| Dow Corning | Dow Corning Fluid | Open | 25.3 |
| | 246 | | |
| Henkel Cognis | Eutanol G | Octyldodecanol | 24.8 |
| Condea Chemie | Isofol 16 | Hexyl Decanol | 24.3 |
| ALZO (ROVI) | Dermol 139 | Isotridecyl 3,5,5 | 24.5 |
| | | Trimethylhexanonanoate | |
| Henkel Cognis | Cetiol PGL | Hexyldecanol (+) Hexyl Decyl | 24.3 |
| | | Laurate | |
| | Cegesoft C24 | Octyl Palmitate | 23.1 |
| Gattefossé | M.O.D. | Octyldodeceyl Myristate | 22.1 |
| | Macadamia Nut | | 22.1 |
| | Oil | | |
| Bayer AG, | Silicone oil VP | Phenyl Trimethicone | 22.7 |
| Dow Corning | 1120 | | |
| CONDEA Chemie | Isocarb 12 | Butyl Octanoic Acid | 22.1 |
| Henkel Cognis | Isopropyl stearate | Isopropyl Stearate | 21.9 |
| WITCO, | Finsolv TN | C12-15 Alkyl Benzoate | 21.8 |
| Goldschmidt | | | |
| Dr. Straetmans | Dermofeel BGC | Butylene Glycol | 21.5 |
| | | Caprylate/Caprate | |
| Unichema | Miglyol 812 | Caprylic/Capric Triglyceride | 21.3 |
| Huels | | | |
| Trivent (via S. | Trivent OCG | Tricaprylin | 20.2 |
| Black) | | | |
| ALZO (ROVI) | Dermol 866 | PEG,, Diethylhexanoate/ | 20.1 |
| | | Diisononanoate/Ethylhexyl | |
| | | Isononanoate | |

Compositions according to the invention can be formulated such that they develop finely-bubbled foams during their preparation—for example during stirring or during homogenization.

Such self-foaming or foam-like cosmetic or dermatological preparations for the purposes of the present invention are characterized in that they comprise 1 to 90% by volume, based on the total volume of the preparation, of at least one gas chosen from the group consisting of air, oxygen, nitrogen, helium, argon, nitrous oxide (N₂O) and carbon dioxide (CO₂).

Self-foaming or foam-like cosmetic or dermatological preparations for the purposes of the present invention can advantageously comprise inorganic particulate hydrophobic or hydrophobicized or oil-absorbing solid-body substances or inorganic gel formers.

Advantageous inorganic particulate hydrophobic or hydrophobicized or oil-absorbing solid-body substances may, for example, be chosen from the group

of inorganic fillers (such as talc, kaolin, zeolites, boron nitride),

of inorganic pigments based on metal oxides or other metal compounds which are sparingly soluble or insoluble in water (in particular oxides of titanium, zinc, iron, manganese, aluminum, cerium),

of inorganic pigments based on silicon oxides (such as, in particular, the grades Aerosil-200, Aerosil 200 V),

of silicate derivatives (such as sodium silicoaluminates or fluoro magnesium silicates (submica grades), calcium aluminum borosilicates). Preference is given here in particular to silica dimethyl silylate (Aerosil® R972).

Aerosils [(fumed silica)=silicon dioxide obtained by thermal decomposition of ethyl silicate)] are highly disperse silicas with an often irregular shape, whose specific surface area is usually very large (200-400 m²/g) and can be controlled depending on the preparation process.

Aerosils to be used particularly advantageously according to the invention are obtainable, for example, under the trade names: Aerosil® 130 (Degussa Hüls), Aerosil® 200 (Degussa Hüls), Aerosil® 225 (Degussa Hüls), Aerosil® 300 (Degussa Hüls), Aerosil® 380 (Degussa Hüls), B-6C (Suzuki Yushi), CAB-O-SIL Fumed Silica (Cabot), CAB-O-SIL EH-5 (Cabot), CAB-O-SIL HS-5 (Cabot), CAB-O-SIL LM-130 (Cabot), CAB-O-SIL MS-55 (Cabot), CAB-O-SIL M-5 (Cabot), E-6C (Suzuki Yushi), Fossil Flour MBK (MBK), MSS-500 (Kobo), Neosil CT 11 (Crosfield Co.), Ronasphere (Rona/EM Industries), Silica, Anhydrous 31 (Whittaker, Clark & Daniels) Silica, Crystalline 216 (Whittaker, Clark & Daniels), Silotrat-1 (Vevy), Sorbosil AC33 (Crosfield Co.), Sorbosil AC 35 (Crosfield Co.), Sorbosil AC 37 (Crosfield Co.), Sorbosil AC 39 (Crosfield Co.), Sorbosil AC77 (Crosfield Co.), Sorbosil TC 15 (Crosfield Co.), Spherica (Ikeda), Spheriglass (Potters-Ballotini), Spheron L-1500 (Presperse), Spheron N-2000 (Presperse), Spheron P-1500 (Presperse), Wacker HDK H 30 (Wacker-Chemie), Wacker HDK N 20 (Wacker-Chemie), Wacker HDK P 100H (Wacker Silicones), Wacker HDK N 20P (Wacker-Chemie), Wacker HDK N 25P (Wacker-Chemie), Wacker HDK S 13 (Wacker-Chemie), Wacker HDK T 30 (Wacker-Chemie), Wacker HDK V 15 (Wacker-Chemie), Wacker HDK V 15 P (Wacker Chemie), Zelec Sil (DuPont).

It is also advantageous to use those SiO₂ pigments in which the free OH groups on the surface of the particles have been (completely or partially) organically modified. This gives, for example as a result of the addition of dimethylsilyl groups, silica dimethyl silylate (e.g. Aerosil® R972 (Degussa Hüls), Aerosil® R974 (Degussa Hüls), CAB-O-SIL TS-610 (Cabot), CAB-O-SIL TS-720 (Cabot), Wacker HDK H15 (Wacker-Chemie), Wacker HDK H18 (Wacker-Chemie), Wacker HDK H20 (Wacker-Chemie)). The addition of trimethylsilyl groups gives silica silylate (e.g. Aerosil R 812 (Degussa Hüls), CAB-O-SIL TS-530 (Cabot), Sipernat D 17 (Degussa Hüls), Wacker HDK H2000 (Wacker-Chemie)).

Very advantageous inorganic gel formers can, for example, be chosen from the group of modified or unmodified, naturally occurring or synthetic sheet silicates. Although it is entirely favorable to use pure components, the preparations according to the invention may also advantageously comprise mixtures of different modified or unmodified sheet silicates.

Sheet silicates, which are so-called phyllosilicates, are understood for the purposes of this application as meaning silicates and alumosilicates in which the silicate or aluminate units, respectively, are joined together via three Si—O or Al—O bonds and form a waved sheet or layer structure. The fourth Si—O or Al—O valence is saturated by cations. There are relatively weak electrostatic interactions, e.g. hydrogen bridge bonds, between the individual layers. The layer structure is consequently defined largely by strong covalent bonds.

The stoichiometery of the sheet silicates is (Si₂O₅²⁻) for pure silicate structures and (AlₘSi²⁻ₘO₅(^{2+m})⁻) for alumosilicates, where m is a number greater than zero and less than 2.

If no pure silicates are present, but alumosilicates, it should be taken into consideration that each Si⁴⁺ group replaced by Al³⁺ requires a further singly charged cation to neutralize the charge.

The charge balance is preferably balanced by H⁺, alkali metal or alkaline earth metal ions. Aluminum as counterion is also known and advantageous. In contrast to the alumosilicates, these compounds are called aluminum silicates. “Aluminum alumosilicates”, in which aluminum is present both in the silicate network, and also as counterion, are also known and in some cases advantageous for the present invention.

Sheet silicates are well documented in the literature, e.g. in the “Lehrbuch der Anorganischen Chemie” [Textbook of inorganic chemistry], A. F. Hollemann, E. Wiberg and N. Wiberg, 91st-100th edition, Walter de Gruyter—Verlag 1985, passim, and also “Lehrbuch der Anorganischen Chemie” [Textbook of inorganic chemistry], H. Remy, 12th edition, Akademische Verlagsgesellschaft, Leipzig 1965, passim. The layer structure of montmorillonite can be found in Römpps Chemie-Lexikon, Franckh'sche Verlagshandlung W. Keller & Co., Stuttgart, 8th edition, 1985, p. 2668 f.

Examples of sheet silicates are:

| | |
|---|---|
| Montmorilonite | Na_{0.33}((Al_{1.67}Mg_{0.33})(OH)₂(Sᵢ₄O₁₀)) |
| often simplified | Al₂O₃*4SiO₂*H₂O*nH₂O or Al₂[(OH)₂/Si₄O₁₀].n H₂O |
| to Kaolinite | Al₂(OH)₄(Si₂O₅) |
| Ilite | (K,H₃O)_{y}(Mg₃(OH)₂(Si_{4-y}Al_{y}O₁₀)) |
| and | (K,H₃O)_{y}(Al₂(OH)₂(Si_{4-y}Al_{y}O₁₀)) |
| | where y = 0.7-0.9 |
| Beidellite | (Ca,Na)_{0.3}(Al₂(OH)₂(Al_{0.5}Si_{3.5}O₁₀)) |
| Nontronite | Na_{0.33}(Fe₂(OH)₂(Al_{0.33}S_{13.67}O₁₀)) |
| Saponite | (Ca,Na)_{0.33}(Mg,Fe)₃(OH)₂(Al_{0.33}Si_{3.67}O₁₀)) |
| Hectorite | Na_{0.33}((Mg,Li)₃(OH,F)₂(Si₄O₁₀)) |

Montmorillonite represents the main mineral of the naturally occurring bentonites.

Very advantageous inorganic gel formers for the purposes of the present invention are aluminum silicates, such as the montmorillonites (bentonites, hectorites and derivatives thereof, such as quaternium-18 bentonite, quaternium-18 hectorite, stearalkonium bentonite and stearalkonium hectorite) or however magnesium aluminum silicates (Veegum® grades), and sodium magnesium silicates (Laponite® grades).

Montmorillonites represent clay minerals which belong to the dioctahedral smectites, and are masses which swell in water, but do not become plastic. The layer packets in the three-layer structure of the montmorillonites can swell as the result of reversible incorporation of water (in a 2- to 7-fold amount) and other substances such as, for example, alcohols, glycols, pyridine, α-picoline, ammonium compounds, hydroxy-aluminosilicate ions etc.

The chemical formula given above is only approximate; since montmorillonite has a large capacity for ion exchange, Al can be replaced by Mg, Fe²⁺, Fe²⁺, Zn, Pb, Cr, and also Cu and others. The resulting negative charge of the octahedral layers is balanced by cations, in particular Na⁺ (sodium montmorillonite) and Ca²⁺ (calcium montmorillonite is only swellable to a very small degree) in interlayer positions.

Synthetic magnesium silicates or bentonites advantageous for the purposes of the present invention are sold, for example, by Süd-Chemie under the trade name Optigel®.

An aluminum silicate advantageous for the purposes of the present invention is sold, for example, by R.T. Vanderbilt Comp., Inc., under the trade name Veegum®. The various Veegum® grades, which are all advantageous according to the invention, are characterized by the following compositions

| | (regular grade) | HV | K | HS | S-728 |
|---|---|---|---|---|---|
| SiO₂ | 55.5 | 56.9 | 64.7 | 69.0 | 65.3 |
| MgO | 13.0 | 13.0 | 5.4 | 2.9 | 3.3 |
| Al₂O₃ | 8.9 | 10.3 | 14.8 | 14.7 | 17.0 |
| Fe₂O₃ | 1.0 | 0.8 | 1.5 | 1.8 | 0.7 |
| CaO | 2.0 | 2.0 | 1.1 | 1.3 | 1.3 |
| Na₂O | 2.1 | 2.8 | 2.2 | 2.2 | 3.8 |
| K₂O | 1.3 | 1.3 | 1.9 | 0.4 | 0.2 |
| Ashing loss | 11.1 | 12.6 | 7.6 | 5.5 | 7.5 |

These products swell in water to form viscous gels, which have an alkaline reaction. The organophilization of montmorillonite or bentonites (exchange of the interlayer cations for quaternary alkylammonium ions) produces products (bentones) which are preferably used for dispersion in organic solvents and oils, fats, ointments, paints, coatings and in detergents.

Bentone® is a trade name for various neutral and chemically inert gelling agents which are constructed from long-chain, organic ammonium salts and specific types of montmorillonite.

The following Bentone® grades are sold, for example, by Kronos Titan and are to be used advantageously for the purposes of the present invention: Bentone® 27, an organically modified montmorillonite, Bentone® 34 (dimethyldioctylammonium bentonite), which is prepared in accordance with U.S. Pat. No. 2,531,427 and, because of its lipophilic groups, swells more readily in a lipophilic medium than in water, Bentone® 38, an organically modified montmorillonite, a cream-colored to white powder, Bentone® LT, a purified clay mineral, Bentone® Gel MIO, an organically modified montmorillonite which is supplied as a very fine suspension in mineral oil (SUS-71) (10% bentonite, 86.7% mineral oil and 3.3% wetting agent), Bentone® Gel IPM, an organically modified bentonite which is suspended in isopropyl myristate (10% bentonite, 86.7% isopropyl myristate, 3.3% wetting agent), Bentone® Gel CAO, an organically modified montmorillonite which is taken up in castor oil (10% bentonite, 86.7% castor oil and 3.3% wetting agent), Bentone® Gel Lantrol, an organically modified montmorillonite which, in paste form, is intended for the further processing, in particular for the preparation of cosmetic compositions; 10% bentonite, 64.9 Lantrol (wool wax oil), 22.0 isopropyl myristate, 3.0 wetting agent and 0.1 propyl p-hydroxybenzoate, Bentone® Gel Lan I, a 10% strength Bentone® 27 paste in a mixture of wool wax USP and isopropyl palmitate, Bentone® Gel Lan II, a bentonite paste in pure, liquid wool wax, Bentone® Gel NV, a 15% strength Bentone® 27 paste in dibutyl phthalate, Bentone® Gel OMS, a bentonite paste in Shellsol T., Bentone® Gel OMS 25, a bentonite paste in isoparaffinic hydrocarbons (Idopar® H), Bentone® Gel IPP, a bentonite paste in isopropyl palmitate.

All Bentone grades are to be used advantageously for the purposes of the present invention.

For the purposes of the present invention, self-foaming or foam-like preparations may also advantageously comprise one or more hydrocolloids from one or more of the following groups:

organic, natural compounds, such as, for example, agar agar, carrageen, tragacanth, gum arabic, alginates, pectins, polyoses, guar flour, carob bean flour, starch, dextrins, gelatin, casein,

organic, modified natural substances, such as, for example, carboxymethylcellulose and other cellulose ethers, hydroxyethyl cellulose and hydroxypropyl cellulose and microcrystalline celluose the like,

organic, completely synthetic compounds, such as, for example, polyacrylic and polymethacrylic compounds, vinyl polymers, polycarboxylic acids, polyethers, polyimines, polyamides, polyurethanes.

The cosmetic or dermatological compositions according to the invention can also advantageously be in the form of foamable preparations which, for example, are removed from aerosol containers and thereby foamed. Aerosol containers advantageous according to the invention are spray devices with a filling of the liquid or slurry-like substances, which are under the pressure of a propellant (pressurized gas or aerosol packaging). Such containers can be provided with valves of very different construction which permit the removal of the contents in the form of a foam.

In addition, foamable preparations according to the present invention may advantageously be removed from propellant gas-free, mechanically operated pump atomizers (pump dispensers). Particularly advantageous for the purposes of the present invention are pump systems which work without pressurized gas, but with a filter which brings about special swirling.

The cosmetic or dermatological preparations according to the invention can also preferably be present in after-foamable form and be removed, for example, from two-chamber aerosol containers and be applied to the skin. Packaging means advantageous according to the invention are containers in which there is one chamber with a filling of the liquid or slurry-like preparations under the pressure of a primary propellant located in a second chamber. Such containers can be equipped with valves of very different construction which permit the removal of the contents of the first chamber in the form of an emulsion or gel in any position—even with the valve pointing downward.

An advantageous embodiment are BiCan® aerosol containers in which the product is enclosed in a flexible bag made of metal or plastic within the can.

After-foaming compositions according to the invention represent unfoamed, i.e. directly after exiting an aerosol container, two- or multiphase systems—generally emulsions. They can be made into foams by gentle rubbing, for example in the hands or during application and rubbing on the skin, but also by stirring or other foaming operations.

Moreover, it has surprisingly been found that in the case of the use of (secondary) propellants, particularly advantageously of propellants soluble in the optionally present oil phase, thus, for example, customary propane/butane mixtures, the preparations according to the invention are not simply sprayed as aerosol droplets, but develop into finely-bubbled, rich foams as soon as such systems containing such (secondary) propellants experience decompression.

In the case of the use of hydrocarbons or mixtures thereof having 4 or 5 carbon atoms, in particular isobutane, n-pentane and isopentane, as (secondary) propellant, it is possible to delay the automatic foaming after exiting from the pressurized packaging.

As a result of the evaporation of the secondary propellant in the applied cosmetic product, heat is additionally withdrawn from the skin and a pleasant cooling effect is achieved. Such after-foaming preparations are therefore likewise regarded as advantageous embodiments of the present invention with independent inventive step.

Suitable pressurized gas containers for the purposes of the present invention are primarily cylindrical vessels made of metal (aluminum, tinplate, contents <1 000 ml), protected or shatter-resistant glass or plastic (contents <220 ml) or shattering glass or plastic (contents <150 ml), in the choice of which compressive strength and breaking strength, corrosion resistance, ease of filling, or ease of sterilizing etc., but also esthetic aspects, handlability, printing properties, etc. play a role. The maximum permissible operating pressure of spray cans made of metal at 50° C. is 12 bar and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminum and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) or polyethylene terephthalate (PET) on the inside of the cans are also advantageous, in particular for cans made of tinplate.

The pressurized gas containers are usually single-part or two-part, but in most cases three-part cylindrical, conical or differently shaped. If plastics are used as the spray container material, then these should be resistant to chemicals and the sterilization temperature, gas-tight, impact-resistant and stable to internal pressures in excess of 12 bar. In principle, polyacetals and polyamides are suitable for spray container purposes.

The internal construction of the spray cans and the valve construction are many and varied, depending on the intended use and the physical nature of the ingredient—e.g. whether it is in the form of a two-phase or three-phase system—and can be determined by the person skilled in the art by simple trial and error without inventive activity. For suitable variants, reference may be made to the “Aerosol Technologie Handbuch der Aerosol-Verpackung” [Aerosol Technology Handbook of Aerosol Packaging] (Wolfgang Tauscher, Melcher Verlag GmbH Heidelberg/Munich, 1996).

Valves which are advantageous according to the invention can be designed with or without riser tube. The individual components from which valves according to the invention are usually constructed preferably consist of the following materials:

Disk: tinplate: uncoated, gold- or clear-coated, film-laminated (PE, PP or PET) aluminum: uncoated, silver- or gold-coated, different coating variants, Stoner-Mudge design

Seal: natural or synthetic elastomers or thermoplastic (sleeve gaskets, film-laminated made of PE or PP) internal and external seals, e.g. made of perbunan, buna, neoprene, butyl, CLB, LDPE, viton, EPDM, chlorobutyl, bromobutyl or diverse compounds

Cone: PA, POM, brass and diverse special materials,

standard bores (e.g.: 0.25 to 0.70 mm or 2×0.45 to 2×1.00 mm),

various shaft diameters

Spring: metal, particularly preferably V2A, stainless steel;

plastic and also elastomer

Casing: standard and impact

VPH bores, RPT bores or slit for overhead applications

materials: e.g. polyacetal, PA, PE, POM and the like

Riser tube: plastic (polymer resin), e.g. PE, PP, PA or polycarbonate

Advantageous spray heads for the purposes of the present invention are, for example, foaming heads for upright use (hold can vertically) or foam heads for overhead application using one or more channels.

Suitable (secondary) propellants are the customary “classic” readily volatile, liquefied propellant gases, such as, for example, dimethyl ether (DME) and linear or branched-chain hydrocarbons with two to five carbon atoms (such as, in particular, ethane, propane, butane, isobutane and pentane), which can be used on their own or in a mixture with one another.

Compressed air, and also other gases which are under pressure, such as air, oxygen, nitrogen, hydrogen, helium, krypton, xenon, radon, argon, nitrous oxide (N₂O) and carbon dioxide (CO₂) are also advantageously to be used for the purposes of the present invention as propellant gases (either on their own or in any desired mixtures with one another).

The person skilled in the art is naturally aware that there are other propellant gases which are nontoxic per se and which would be suitable in principle for realizing the present invention in the form of aerosol preparations, but which nevertheless should be omitted due to an unacceptable impact on the environment or other accompanying circumstances, in particular halogenated (substituted by fluorine, chlorine, bromine, iodine or astatine) hydrocarbons, such as, for example, fluorocarbons and chlorofluorocarbons (CFCs).

For the purposes of the present invention, said gases can in each case be used individually or in any desired mixtures with one another.

For the purposes of the present invention, the volume fraction of propellant gas is advantageously chosen from the range from 0.1 to 30% by volume, based on the total volume of filler material and propellant gas (corresponding to a volume fraction of from 70 to 99.9% by volume of filler material).

A particularly preferred propellant gas for the purposes of the present invention is carbon dioxide. Foams obtainable from preparations according to the invention which comprise carbon dioxide as one or the active ingredient are particularly advantageous.

Particularly advantageous, finely creamy and rich foams are obtainable when the preparations according to the invention are foamed using linear or branched-chain, halogenated or nonhalogenated hydrocarbons. Very particularly advantageous foams are obtainable by foaming the preparations according to the invention with carbon dioxide, oxygen, compressed air, helium, krypton, xenon, radon, argon or nitrogen (either on their own or in any desired mixtures with one another).

The cosmetic or dermatological preparations according to the invention can have the customary composition. Particularly advantageous for the purposes of the present invention are skincare preparations: they can be used for cosmetic or dermatological light protection, and also for the treatment of the skin or of the hair and as a make-up product in decorative cosmetics. A further advantageous embodiment of the present invention consists in after-sun products.

Depending on their formulation, cosmetic or topical dermatological compositions for the purposes of the present invention can, for example, be used as skin protection cream, day or night cream etc. It is optionally possible and advantageous to use the compositions according to the invention as bases for pharmaceutical formulations.

Just as emulsions of liquid and solid consistency are used as cosmetic cleansing lotions or cleansing creams, the preparations according to the invention can also represent “cleansing foams”, which can be used, for example, for the removal of make-up or as a mild washing foam—optionally also for blemished skin. Such cleansing foams can also advantageously be applied as so-called “rinse off” preparations, which are rinsed off from the skin following application.

The cosmetic or dermatological preparations according to the invention can also advantageously be in the form of a foam for the care of hair or the scalp, in particular a foam for arranging the hair, a foam which is used while blow-drying the hair, a styling and treatment foam.

For use, the cosmetic and dermatological preparations according to the invention are applied to the skin or the hair in an adequate amount in the manner customary for cosmetics.

It is advantageous for the purposes of the present invention when the preparations according to the invention comprise substances which further improve the stability of the foamed preparations, increase the introduction of gases or improve the cosmetic properties of the foams according to the invention. Such substances are also referred to below as “foam boosters”.

For the purposes of the present invention, foam boosters may advantageously be chosen from the group of copolymers which are characterized by the following structural formula (I)

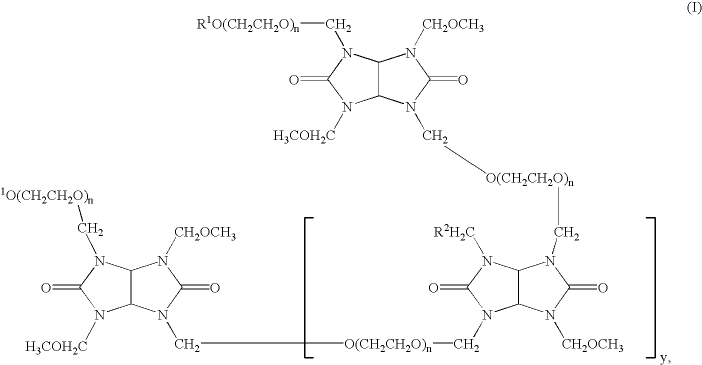

in which

R¹ is a branched or unbranched, saturated or unsaturated alkyl radical having 4 to 40 carbon atoms,

R²=—OCH₃ or —O(CH₂CH₂O)ₓR¹,

x is an integer from 1 to 100,

n is an integer from 100 to 250 and

y is on average 2 or 3.

Particularly advantageous foam boosters for the purposes of the present invention are those copolymers for which n is an integer from 150 to 200. It is particularly advantageous for the purposes of the present invention if, moreover, R¹ is a branched or unbranched, saturated or unsaturated alkyl radical having 8 to 12 carbon atoms.

It is also advantageous for the purposes of the present invention when the average molar mass of the copolymers is between 30 000 and 50 000.

Very particularly advantageous according to the invention are PEG-180/octoxynol-40/tetramethoxymethylglycoluril copolymers where R²=—O(CH₂CH₂O)₄₀C₈H₁₇ and n=180.

Also particularly advantageous according to the invention are PEG-180/Laureth-50/tetramethoxymethylglycoluril copolymers where R²=—O(CH₂CH₂O)₅₀C₁₂H₂₅ and n=180.

Also particularly advantageous according to the invention is polyether-1.

Waxes or lipids which are solid or semisolid at room temperature are also advantageous foam boosters for the purposes of the present invention.

Waxes or lipids which are solid or semisolid at room temperature for the purposes of the present invention are compounds which are characterized in that, together with the other oil components of the preparations according to the invention (such as, for example, polar, liquid compounds, UV filters and solvents thereof etc.), they form a mass which is spreadable or flowable at room temperature and which has a viscosity of more than 500 mPa·s at 20° C.

Advantageous waxes or lipids according to the invention are, for example, paraffin hydrocarbons, synthetic and semisynthetic waxes or wax esters, and plant waxes and mixtures thereof with melting points or solidification points of from 25 to 125° C.

Advantageous waxes according to the invention are also those listed below:

| Class | Subgroup | Examples |
|---|---|---|
| Natural | Plant waxes | Candelilla wax, carnauba wax, Japan |
| waxes | | wax, esparto grass wax, cork wax, |
| | | guaruma wax, ricegerm oil wax, sugar |
| | | cane wax, ouricury wax, montan wax |
| | Animal waxes | Beeswax, shellac wax, spermaceti, |
| | | lanolin (wool wax), uropygial grease |
| | Mineral waxes | Ceresin, ozokerite (earth wax) |
| Chemically | Hard waxes | Montan ester waxes, sasol waxes, |
| modified | | hydrogenated jojoba waxes |
| waxes | | |
| Synthetic | | Polyalkylene waxes, polyethylene glycol |
| waxes | | waxes |

The cosmetic and dermatological preparations according to the invention can comprise cosmetic auxiliaries, as are customarily used in such preparations, e.g. preservatives, preservative assistants, bactericides, perfumes, dyes, pigments which have a coloring effect, moisturizing or humectant substances, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin), iodopropyl butylcarbamates (e.g. those available under the trade names Koncyl-L, Koncyl-S and Konkaben LMB from Lonza), parabens, phenoxyethanol, ethanol, benzoic acid and the like. According to the invention, the preservative system usually also advantageously comprises preservative assistants, such as, for example, octoxyglycerol, glycine soya, etc.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic or dermatological applications.

For the purposes of the present invention, water-soluble antioxidants, such as, for example, vitamins, e.g. ascorbic acid and derivatives thereof, can be used particularly advantageously.

Preferred antioxidants are also vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

It is particularly advantageous when the cosmetic preparations according to the present invention comprise cosmetic or dermatological active ingredients, preferred active ingredients being antioxidants which can protect the skin against oxidative stress.

Further advantageous active ingredients for the purposes of the present invention are natural active ingredients or derivatives thereof, such as, for example, alpha-lipoic acid, phytoene, D-biotin, coenzyme Q10, alpha-glycosylrutin, carnitine, carnosine, natural or synthetic isoflavonoids, creatine, taurine or β-alanine.

Formulations according to the invention which comprise, for example, known anti-wrinkle active ingredients, such as flavone glycosides (in particular α-glycosylrutin), coenzyme Q10, vitamin E and derivatives and the like are particularly advantageously suitable for the prophylaxis and treatment of cosmetic or dermatological changes in the skin, as arise, for example, during skin aging (such as, for example, dryness, roughness and the formation of dryness wrinkles, irritation, reduced refatting (e.g. after washing), visible vascular dilations (telangiectases, couperosis), sagging and formation of lines and wrinkles, local hyperpigmentation, hypopigmentation and abnormal pigmentation (e.g. age spots), increased susceptibility to mechanical stress (e.g. cracking) and the like). They are also advantageously suitable against the appearance of dry or rough skin.

Surprisingly, selected formulations according to the invention can also have an anti-wrinkle effect or considerably increase the effect of known anti-wrinkle active ingredients. Accordingly, formulations for the purposes of the present invention are particularly advantageously suitable for the prophylaxis and treatment of cosmetic or dermatological skin changes, as arise, for example, during skin aging. They are also advantageously suitable for combating the appearance of dry or rough skin.

In one particular embodiment, the present invention thus relates to products for the care of skin aged in a natural manner, and for the treatment of the secondary damage of photoaging, in particular the phenomena listed above.

The water phase of the preparations according to the invention can advantageously comprise customary auxiliaries, such as, for example, alcohols, in particular those of low carbon number, preferably ethanol, isopropanol, diols or polyols of low carbon number, and ethers thereof, preferably polyethylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilizers, electrolytes and moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL)) or have a beneficial effect on the hydration of the horny layer.

Advantageous moisturizers for the purposes of the present invention are, for example, glycerol, lactic acid, pyrrolidone carboxylic acid and urea. In addition, it is particularly advantageous to use polymeric moisturizers from the group of water-soluble, water-swellable, or water-gellable polysaccharides. Particularly advantageous are, for example, hyaluronic acid, chitosan, or a fucose-rich polysaccharide which is listed in the Chemical Abstracts under the registry number 178463-23-5 and is available, for example, under the name Fucogel® 1000 from SOLABIA S.A.

The cosmetic or dermatological preparations according to the invention can comprise dyes or color pigments, particularly when they are in the form of decorative cosmetics. The dyes or color pigments can be chosen from the corresponding positive list of the Cosmetics Directive or the EC list of cosmetic colorants. In most cases they are identical to the dyes approved for foodstuffs. Advantageous color pigments are, for example, titanium dioxide, mica, iron oxides (e.g. Fe₂O₃, Fe₃O₄, FeO(OH)) and tin oxide. Advantageous dyes are, for example, carmine, Prussian blue, chromium oxide green, ultramarine blue, or manganese violet. It is particularly advantageous to choose the dyes or color pigments from the Rowe Colour Index, 3rd edition, Society of Dyers and Colourists, Bradford, England, 1971.

If the formulations according to the invention are in the form of products which are used on the face, it is favorable to choose one or more substances from the following group as dye: 2,4-dihdyroxyazobenzene, 1-(2′-chloro-4′-nitro-1′-phenylazo)-2-hydroxynaphthalene, Ceres red, 2-(sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxypyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalonedisulfonic acid, aluminum salt of indigodisulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochineal.

Also advantageous for the purposes of the present invention are formulations with a content of pearlescent pigments. Preference is given in particular to the types of pearlescent pigments listed below:

1. Natural pearlescent pigments, such as, for example

“pearl essence” (guanine/hypoxanthin mixed crystals from fish scales) and

“mother of pearl” (ground mussel shells)

2. Monocrystalline pearlescent pigments, such as, for example, bismuth oxychloride (BiOCl)

3. Layer-substrate pigments: e.g. mica/metal oxide

Bases for pearlescent pigments are, for example, pulverulent pigments or castor oil dispersions of bismuth oxychloride or titanium dioxide, and bismuth oxychloride or titanium dioxide on mica. The luster pigment listed under CIN 77163, for example, is particularly advantageous.

Also advantageous are, for example, the following types of pearlescent pigment based on mica/metal oxide:

| Group | Coating/layer thickness | Color |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Prussian blue | deep blue |
| | TiO₂/carmine | red |

Particular preference is given, for example, to the pearlescent pigments obtainable from Merck under the trade names Timiron, Colorona, and Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines are advantageous.

It may, moreover, be advantageous to dispense completely with a substrate such as mica. Particular preference is given to iron pearlescent pigments prepared without the use of mica. Such pigments are obtainable, for example, under the trade name Sicopearl Copper 1000 from BASF.

In addition, also particularly advantageous are effect pigments which are obtainable under the trade name Metasomes Standard/Glitter in various colors (yellow, red, green, blue) from Flora Tech. The glitter particles here are present in mixtures with various auxiliaries and dyes (such as, for example, the dyes with the Colour Index (CI) numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from, for example, 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of UV protectants. Thus, for example, UV-A and/or UV-B filter substances are usually incorporated into day creams or make-up products. UV protectants, like antioxidants, and, if desired, preservatives, also constitute effective protection of the preparations themselves against spoilage. Also favorable are cosmetic and dermatological preparations in the form of a sunscreen.

Accordingly, for the purposes of the present invention, besides comprising one or more UV filter substances according to the invention, the preparations additionally preferably comprise at least one further UV-A and/or UV-B filter substance. The formulations may, although not necessarily, optionally also comprise one or more organic or inorganic pigments as UV filter substances which may be present in the water or oil phase.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides and other metal compounds which are insoluble or sparingly soluble in water, in particular oxides of titanium (TiO₂), zinc (ZnO), iron (e.g. Fe₂O₃). zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and also the sulfate of barium (BaSO₄).

The pigments can, for the purposes of the present invention, also advantageously be used in the form of commercially available oily or aqueous predispersions. Dispersion auxiliaries and/or solubilization promoters may advantageously be added to these predispersions.

According to the invention, the pigments may advantageously be surface-treated (“coated”), the intention being to form or retain, for example, a hydrophilic, amphiphilic, or hydrophobic character. This surface treatment can consist in providing the pigments with a thin hydrophilic or hydrophobic inorganic or organic layer by processes known per se. The various surface coatings may also comprise water for the purposes of the present invention.

Inorganic surface coatings for the purposes of the present invention may consist of aluminum oxide (Al₂O₃), aluminum hydroxide Al(OH)₃, and aluminum oxide hydrate (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings can arise on their own, in combination, or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicone (a mixture of dimethylpolysiloxane with an average chain length of from 200 to 350 dimethylsiloxane units and silica gel) or alginic acid. These organic surface coatings may be present on their own, in combination, or in combination with inorganic coating materials.

Zinc oxide particles suitable according to the invention and predispersions of zinc oxide particles are obtainable under the following trade names from the companies listed:

| Trade name | Coating | Manufacturer |
|---|---|---|
| Z-Cote HP1 | 2% dimethicone | BASF |
| Z-Cote | / | BASF |
| ZnO NDM | 5% dimethicone | H&R |

Suitable titanium dioxide particles and predispersions of titanium dioxide particles are obtainable under the following trade names from the companies listed:

| Trade name | Coating | Manufacturer |
|---|---|---|
| MT-100TV | aluminum hydroxide/stearic | Tayca |
| | acid | Corporation |
| MT-100Z | aluminum hydroxide/stearic | Tayca |
| | acid | Corporation |
| Eusolex T-2000 | alumina/simethicone | Merck KgaA |
| Titanium dioxide | octyltrimethylsilane | Degussa |
| T805 | | |
| (Uvinul TiO₂) | | |

An advantageous organic pigment for the purposes of the present invention is 2,2′-methylenebis(6-(2H-benzotriazol-2-yl)-4-(1,1,3,3-tetramethylbutyl)phenol) [INCI: Bisoctyltriazole], which is available under the trade name Tinosorb® M from CIBA-Chemikalien GmbH.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex® 9020.

Further advantageous UV-A filter substances for the purposes of the present invention are hydroxybenzophenones, which are characterized by the following structural formula:

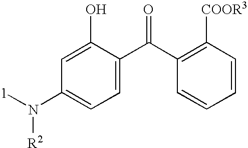

in which

R¹ and R², independently of one another, are hydrogen, C₁-C₂₀-alkyl, C₃-C₁₀-cycloalkyl or C₃-C₁₀-cycloalkenyl, where the substituents R¹ and R², together with the nitrogen atom to which they are bonded, can form a 5-membered or 6-membered ring and

R³ is a C₁-C₂₀-alkyl radical.

A particularly advantageous hydroxybenzophenone for the purposes of the present invention is hexyl 2-(4′-diethylamino-2′-hydroxybenzoyl)benzoate (also: aminobenzophenone), which is characterized by the chemical structural formula

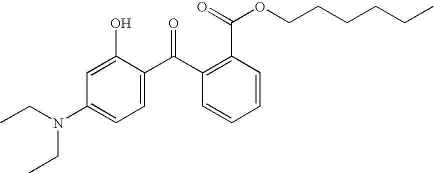

Advantageous further UV filter substances for the purposes of the present invention are sulfonated, water-soluble UV filters, such as, for example,

phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;

salts of 2-phenylbenzimidazole-5-sulfonic acid, such as its sodium, potassium or its triethanolammonium salt, and the sulfonic acid itself with the INCI name Phenylbenzimidazole Sulfonic Acid (CAS No. 27503-81-7), which is available, for example, under the trade name Eusolex 232 from Merck or under Neo Heliopan Hydro from Haarmann & Reimer;

1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethane sulfonic acid) and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;

sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention are also so-called broadband filters, i.e. filter substances which absorb both UV-A and UV-B radiation.

Advantageous broadband filters or UV-B filter substances are, for example, triazine derivatives, such as, for example,

2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA Chemikalien GmbH;

dioctylbutylamidotriazone (INCI: Dioctylbutamidotriazone), which is available under the trade name UVASORB HEB from Sigma 3V;

tris(2-ethylhexyl) 4,4′,4″-(1,3,5-triazine-2,4,6-triyltriimino)trisbenzoate, synonym: 2,4,6-tris[anilino(p-carbo-2′-ethyl-1′-hexyloxy)]-1,3,5-triazine (INCI: Octyl Triazone), which is sold by BASF Aktiengesellschaft under the trade name UVINUL® T 150.

An advantageous broadband filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

A further advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The UV filter substances may be oil-soluble or water-soluble.

Advantageous oil-soluble filter substances are, for example:

3-benzylidenecamphor derivatives, preferably 3-(4-methylbenzylidene)camphor, 3-benzyl idenecamphor;

4-aminobenzoic acid derivatives, preferably 2-ethylhexyl 4-(dimethylamino)benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate;

esters of cinnamic acid, preferably 2-ethylhexyl 4-methoxycinnamate, isopentyl 4-methoxycinnamate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone; and

UV filters bonded to polymers.

Advantageous water-soluble filter substances are, for example: sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light protection filter substance to be used advantageously according to the invention is ethylhexyl 2-cyano-3,3-diphenylacrylate (octocrylene), which is obtainable from BASF under the name Uvinul® N 539.

The list of specified UV filters which may be used for the purposes of the present invention is not of course intended to be limiting.

Particularly advantageous preparations for the purposes of the present invention which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulfonic acid, its salts, 1,4-di(2-oxo-10-sulfo-3-bornylidenemethyl)benzene and salts thereof, 2,4-bis{[4-(2-ethylhexyloxy)-2-hydroxy]phenyl}-6-(4-methoxyphenyl)-1,3,5-triazine, and hydroxybenzonephenone, in each case individually or in any combinations with one another.

The examples below serve to illustrate the present invention without limiting it. Unless stated otherwise, all amounts, fractions and percentages are based on the weight and the total amount or on the total weight of the preparations.

### FORMULATION EXAMPLES

Example 1 (foam-like O/W cream):

| Emulsion I | % by wt. | % by vol. |
|---|---|---|
| Stearic acid | 5.00 | |
| Cetyl alcohol | 5.50 | |
| PEG-40 stearate | 8.50 | |
| Talc | 2.00 | |
| SiO₂ | 2.00 | |
| Magnesium aluminum silicate | 0.50 | |
| Paraffin oil | 5.00 | |
| Isohexadecane | 2.00 | |
| Cyclomethicone | 7.00 | |
| Dimethicone/ | 2.00 | |
| vinyl dimethicone crosspolymer | | |
| Glycerol | 5.00 | |
| Ceresin | 4.00 | |
| Sodium hydroxide | q.s. | |
| Preservative | q.s. | |
| Perfume | q.s. | |
| Water, demineralized | ad 100.00 | |
| pH adjusted to 6.5-7.5 | | |
| Emulsion I | | 70 |
| Gas (nitrogen) | | 30 |

Predispersion of the inorganic gel former and swelling of the hydrocolloid, and of the polymer with stirring in the water phase. Combining of the fatty phase heated to 75° C. with the water phase heated to 70° C. Addition of the particulate hydrophobic, hydrophobicized solid-body substances with stirring. Homogenization by means of a toothed-wheel dispersing machine (rotor-stator principle) at 65° C. 45 min stirring with gassing with nitrogen at 0.7 bar and cooling. Addition of the additives at 30° C. (perfume, active ingredients). Homogenization by means of a toothed-wheel dispersing machine (rotor-stator principle) at 27° C.

### Example 2

### Foam-Like O/W Lotion

| Emulsion II | % by wt. | % by vol. |
|---|---|---|
| Stearic acid | 4.00 | |
| Myristyl alcohol | 1.50 | |
| Cetylstearyl alcohol | 0.50 | |
| PEG-100 stearate | 4.00 | |
| Kaolin | 0.05 | |
| Hydroxyethylcellulose | 0.05 | |
| Magnesium aluminum silicate | 0.20 | |
| Mineral oil | 8.00 | |
| Cyclomethicone | 6.00 | |
| Dimethicone | 3.00 | |
| PEG-180/laureth-50/TMMG copolymer | 0.50 | |
| Glycerol | 3.00 | |
| Aminobenzophenone | 2.00 | |
| Hydrogenated cocoglyceride | 5.00 | |
| Sodium hydroxide | q.s. | |
| Preservative | q.s. | |
| Perfume | q.s. | |
| Water, demineralized | ad 100.00 | |
| pH adjusted to 5.0-6.5 | | |
| Emulsion II | | 50 |
| Gas (carbon dioxide) | | 50 |

Predispersion of the inorganic gel former and swelling of the hydrocolloid, and of the polymer with stirring in the water phase. Combining of the fatty phase heated to 80° C. with the water phase heated to 72° C. Addition of the particulate hydrophobic, hydrophobicized solid-body substances with stirring. Homogenization by means of a toothed-wheel dispersing machine (rotor-stator principle) at 65° C. 45 min stirring with gassing with carbon dioxide at 1.2 bar and cooling. Addition of the additives at 30° C. (perfume). Homogenization by means of a toothed-wheel dispersing machine (rotor-stator principle) at 30° C.

### Example 3

### Foam-Like O/W Lotion

| Emulsion III | % by wt. | % by vol. |
|---|---|---|
| Stearic acid | 2.00 | |
| Cetylstearyl alcohol | 2.50 | |
| PEG-30 stearate | 3.00 | |
| Aluminum starch octenylsuccinate | 3.00 | |
| Talc | 0.50 | |
| Polyurethane | 0.10 | |
| Magnesium silicate | 0.10 | |
| PEG-180/octoxynol-40/TMMG copolymer | 0.25 | |
| Cyclomethicone | 5.00 | |
| Dimethicone | 1.00 | |
| Phenyl trimethicone | 1.00 | |
| Dimethicone/ | 1.00 | |
| vinyl dimethicone crosspolymer | | |
| Cetyl palmitate | 1.00 | |
| Cera microcristillina | 1.00 | |
| Hydrogenated polyisobutene | 10.00 | |
| Citric acid | 0.10 | |
| Aminobenzophenone | 1.20 | |
| Glycerol | 5.00 | |
| Perfume, preservative | q.s. | |
| Sodium hydroxide | q.s. | |
| Dyes etc. | q.s. | |
| Water | ad 100.00 | |
| pH adjusted to 6.0-7.5 | | |
| Emulsion III | | 65 |
| Gas (air) | | 35 |

Predispersion of the inorganic gel former and swelling of the hydrocolloids, and of the polymer with stirring in the water phase. Combining of the fatty phase heated to 80° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in an open reactor up to 30° C. Addition of the additives at 30° C. (perfume, active ingredients). Homogenization by means of a toothed-wheel dispersing machine (rotor-stator principle) at 25° C.

### Example 4

### Foam-Like O/W Sunscreen Cream

| Emulsion IV | % by wt. | % by vol. |
|---|---|---|
| Stearic acid | 1.00 | |
| Cetylstearyl alcohol | 2.00 | |
| PEG-20 stearate | 1.00 | |
| Caprylic/capric triglycerides | 2.00 | |
| Paraffin oil | 10.00 | |
| Cyclomethicone | 5.00 | |
| Dimethicone | 0.50 | |
| Dimethicone/ | 1.50 | |
| vinyl dimethicone crosspolymer | | |
| Octyl isostearate | 2.00 | |
| Myristyl myristate | 1.00 | |
| Glycerol | 6.00 | |
| Carboxymethylcellulose | 0.05 | |
| Magnesium aluminum silicate | 0.50 | |
| PEG-180/laureth-50/TMMG copolymer | 0.50 | |
| Talc | 0.50 | |
| Octyl methoxycinnamate | 4.00 | |
| Butylmethoxydibenzoylmethane | 3.00 | |
| Ethylhexyltriazone | 3.00 | |
| Aminobenzophenone | 1.80 | |
| Bis-ethylhexyloxyphenol methoxyphenyl | 2.00 | |
| triazine | | |
| BHT | 0.02 | |
| Na₂H₂EDTA | 0.10 | |
| Perfume, preservative | q.s. | |
| Dyes, etc. | q.s. | |
| Potassium hydroxide | q.s. | |
| Water | ad 100.00 | |
| pH adjusted to 5.0-6.0 | | |
| Emulsion IV | | 35 |
| Gas (argon) | | 65 |

Predispersion of the inorganic gel former and swelling of the hydrocolloids, and of the polymer with stirring in the water phase. Combining of the fatty phase heated to 78° C. with the water phase heated to 75° C. Addition of the particulate hydrophobic, hydrophobicized solid-body substances with stirring. Homogenization by means of a toothed-wheel dispersing machine (rotor-stator principle) at 65° C. 45 min stirring in a Becomix with gassing with argon at 1 bar with cooling to 30° C. Addition of the additives at 30° C. (perfume, active ingredients). Homogenization by means of a toothed-wheel dispersing machine (rotor-stator principle) at 23° C.

### Example 5

### Foamable O/W Cream

| Emulsion V | % by wt. | % by vol. |
|---|---|---|
| Stearic acid | 2.00 | |
| Cetyl alcohol | 1.00 | |
| PEG-30 stearate | 1.00 | |
| Isohexadecanes | 4.00 | |
| Cyclomethicone | 12.00 | |
| Shea butter | 2.50 | |
| Ozokerite | 0.50 | |
| Vitamin E acetate | 1.00 | |
| Retinyl palmitate | 0.20 | |
| Glycerol | 3.00 | |
| BHT | 0.02 | |
| Na₂H₂EDTA | 0.10 | |
| Perfume, preservative | q.s. | |
| Dyes | q.s. | |
| Potassium hydroxide | q.s. | |
| Water | ad 100.00 | |
| pH adjusted to 5.0-7.0 | | |
| Emulsion V | | 90 |
| Gas (dimethyl ether) | | 10 |

### Example 6

### Foamable O/W Lotion

| Emulsion VI | % by wt. | % by vol. |
|---|---|---|
| Stearic acid | 3.00 | |
| Cetylstearyl alcohol | 2.00 | |
| PEG-100 stearate | 2.00 | |
| Paraffin oil | 3.00 | |
| Dimethicone | 1.00 | |
| Cyclomethicone | 2.00 | |
| Vitamin E acetate | 2.00 | |
| Petroleum jelly (petrolatum) | 2.50 | |
| Glycerol | 3.50 | |
| Carboxymethylcellulose | 0.05 | |
| Magnesium aluminum silicate | 0.50 | |
| Kaolin | 0.05 | |
| Talc | 0.50 | |
| Perfume, preservative, dyes etc. | q.s. | |
| Sodium hydroxide | q.s. | |
| Water | ad 100.00 | |
| pH adjusted to 6.0-7.5 | | |
| Emulsion VI | | 95 |
| Gas (propane/butane) | | 5 |

### Example 7

### Foamable O/W Emulsion Make-Up

| Emulsion VII | % by wt. | % by vol. |
|---|---|---|
| Palmitic acid | 3.00 | |
| Cetyl alchol | 3.00 | |
| PEG-100 stearate | 3.00 | |
| Cyclomethicone | 6.00 | |
| Dimethicone | 0.50 | |
| Hydrogenated polyisobutene | 9.50 | |
| Dicaprylyl carbonate | 2.00 | |
| Cetyl ricinoleate | 2.00 | |
| Glycerol | 3.00 | |
| Mica | 1.00 | |
| Iron oxides | 1.00 | |
| Titanium dioxide | 4.50 | |
| Aminobenzophenone | 1.00 | |
| Vitamin A palmitate | 0.10 | |
| Sodium hydroxide | q.s. | |
| Preservative | q.s. | |
| Perfume | q.s. | |
| Water, demineralized | ad 100.00 | |
| pH adjusted to 6.0-7.5 | | |
| Emulsion VII | | 93 |
| Gas (propane/butane) | | 7 |

That which is claimed:

1. A self-foaming, foam-like, after-foaming or foamable cosmetic or dermatological preparation comprising
(i) an emulsifier system comprising
A. at least one emulsifier A selected from the group consisting of completely neutralized, partially neutralized, and unneutralized, branched and unbranched, saturated and unsaturated fatty acids with a chain length of 10 to 40 carbon atoms,
B. at least one emulsifier B selected from the group consisting of polyethoxylated fatty acid esters with a chain length of 10 to 40 carbon atoms and with a degree of ethoxylation of 5 to 100, and
C. at least one coemulsifier C selected from the group consisting of saturated and unsaturated, branched and unbranched fatty alcohols with a chain length of 10 to 40 carbon atoms; and
(ii) up to 30% by weight—based on the total weight of the preparation—of a lipid phase comprising
(a) one or more lipids selected from the group consisting of silicone oils and silicone waxes, and
(b) one or more lipids selected from the group consisting of nonpolar lipids having a polarity of ≧30 mN/m,
where the ratio of (a) to (b) is selected from the range of 1:3 to 3:1.

2. The self-foaming, foam-like, after-foaming, or foamable cosmetic or dermatological preparation as claimed in claim 1, wherein the lipid phase comprises up to 40% by weight—based on the total weight of the lipid phase—of lipids with a polarity of up to 30 mN/m.

3. The self-foaming, foam-like, after-foaming, or foamable cosmetic or dermatological preparation as claimed in claim 1, wherein the weight ratios of emulsifier A to emulsifier B to coemulsifier C (A:B:C) are designated a:b:c, wherein a, b, and c, independently of one another, are from 1 to 5.

4. The self-foaming, foam-like, after-foaming, or foamable cosmetic or dermatological preparation as claimed in claim 3, wherein a, b, and c, independently of one another, are from 1 to 3.

5. The self-foaming, foam-like, after-foaming, or foamable cosmetic or dermatological preparation as claimed in claim 3, wherein a, b, and c are about 1.

6. The self-foaming, foam-like, after-foaming, or foamable cosmetic or dermatological preparation as claimed in claim 1, wherein the total amount of emulsifier A, emulsifier B and coemulsifier C is from 2 to 20% by weight, based on the total weight of the formulation.

7. The self-foaming, foam-like, after-foaming, or foamable cosmetic or dermatological preparation as claimed in claim 1, further comprising at least one hydrophilic emulsifier.

8. The self-foaming, foam-like, after-foaming, or foamable cosmetic or dermatological preparation as claimed in claim 7, wherein the at least one hydrophilic emulsifier is selected from the group consisting of mono-, di-, and tri-fatty acid esters of sorbitan.

9. The self-foaming, foam-like, after-foaming, or foamable cosmetic or dermatological preparation as claimed in claim 7, wherein the total amount of the at least one hydrophilic emulsifier is less than 5% by weight, based on the total weight of the formulation.

10. The self-foaming, foam-like, after-foaming, or foamable cosmetic or dermatological preparation as claimed in claim 1, further comprised of at least one UV filter substance.

11. The self-foaming, foam-like, after-foaming, or foamable cosmetic or dermatological preparation as claimed in claim 10, wherein the total amount of the at least one UV filter substance is present in a total concentration of 0.5 to 10% by weight, based on the total weight of the formulation.

12. The self-foaming, foam-like, after-foaming, or foamable cosmetic or dermatological preparation as claimed in claim 10, wherein the total amount of the at least one UV filter substance is present in a total concentration of 0.5 to 5% by weight, based on the total weight of the formulation.

13. The self-foaming, foam-like, after-foaming, or foamable cosmetic or dermatological preparation as claimed in claim 10, wherein the at least one UV filter substance is a hydroxybenzophenone.

14. The self-foaming, foam-like, after-foaming, or foamable cosmetic or dermatological preparation as claimed in claim 13, wherein the hydroxybenzophenone is hexyl 2-(4′-diethylamino-2′-hydroxybenzoyl)benzoate.

15. The self-foaming or foam-like cosmetic or dermatological preparation as claimed in claim 1, wherein at least one filler material gas is present in a total volume fraction of 10 to 80% by volume, based on the total volume of the preparation.

16. The self-foaming or foam-like cosmetic or dermatological preparation as claimed in claim 15, wherein the at least one filler material gas includes carbon dioxide.

17. A self-foaming, foam-like, after-foaming or foamable cosmetic or dermatological preparation comprising
(i) an emulsifier system comprising
A. at least one emulsifier A selected from the group consisting of completely neutralized, partially neutralized and unneutralized, branched and unbranched, saturated and unsaturated fatty acids with a chain length of 10 to 40 carbon atoms,
B. at least one emulsifier B selected from the group consisting of polyethoxylated fatty acid esters with a chain length of 10 to 40 carbon atoms and with a degree of ethoxylation of 5 to 100, and
C. at least one coemulsifier C selected from the group consisting of saturated and unsaturated, branched and unbranched fatty alcohols with a chain length of 10 to 40 carbon atoms; and
(ii) up to 30% by weight—based on the total weight of the preparation—of a lipid phase comprising
(a) one or more lipids selected from the group consisting of silicone oils and silicone waxes and
(b) one or more lipids selected from the group consisting of nonpolar lipids with a polarity of ≧30 mN/m,
where the ratio of (a) to (b) is selected from the range of 1:3 to 3:1, said preparation being substantially free of mono- or di-glyceryl fatty acid esters.

18. The self-foaming, foam-like, after-foaming, or foamable cosmetic or dermatological preparation as claimed in claim 17, wherein the lipid phase comprises up to 40% by weight—based on the total weight of the lipid phase—lipids with a polarity of up to 30 mN/m.

19. The self-foaming, foam-like, after-foaming, or foamable cosmetic or dermatological preparation as claimed in claim 17, wherein the weight ratios of emulsifier A to emulsifier B to coemulsifier C (A:B:C) are designated a:b:c, wherein a, b, and c, independently of one another, are from 1 to 5.

20. The self-foaming, foam-like, after-foaming, or foamable cosmetic or dermatological preparation as claimed in claim 19, wherein a, b, and c, independently of one another, are from 1 to 3.

21. The self-foaming, foam-like, after-foaming, or foamable cosmetic or dermatological preparation as claimed in claim 19, wherein a, b, and c are about 1.

22. The self-foaming, foam-like, after-foaming, or foamable cosmetic or dermatological preparation as claimed in claim 17, wherein the total amount of emulsifier A, emulsifier B and coemulsifier C is from 2 to 20% by weight, based on the total weight of the formulation.

23. The self-foaming, foam-like, after-foaming, or foamable cosmetic or dermatological preparation as claimed in claim 17, further comprising at least one hydrophilic emulsifier.

24. The self-foaming, foam-like, after-foaming, or foamable cosmetic or dermatological preparation as claimed in claim 23, wherein the at least one hydrophilic emulsifier is selected from the group consisting of mono-, di-, and tri-fatty acid esters of sorbitan.

25. The self-foaming, foam-like, after-foaming, or foamable cosmetic or dermatological preparation as claimed in claim 23, wherein the total amount of the at least one hydrophilic emulsifier is less than 5% by weight, based on the total weight of the formulation.

26. The self-foaming, foam-like, after-foaming, or foamable cosmetic or dermatological preparation as claimed in claim 17, further comprising at least one UV filter substance.

27. The self-foaming or foam-like cosmetic or dermatological preparation as claimed in claim 17, wherein at least one filler material gas is present in a total volume fraction of 10 to 80% by volume, based on the total volume of the preparation.

28. The self-foaming or foam-like cosmetic or dermatological preparation as claimed in claim 27, wherein the at least one filler material gas includes carbon dioxide.

29. A method for preparing foam, comprising foaming
(a) a preparation comprising
(i) an emulsifier system comprising
A. at least one emulsifier A selected from the group consisting of completely neutralized, partially neutralized and unneutralized, branched and unbranched, saturated and unsaturated fatty acids with a chain length of 10 to 40 carbon atoms,
B. at least one emulsifier B selected from the group consisting of polyethoxylated fatty acid esters with a chain length of 10 to 40 carbon atoms and with a degree of ethoxylation of 5 to 100, and
C. at least one coemulsifier C selected from the group consisting of saturated and unsaturated, branched and unbranched fatty alcohols with a chain length of 10 to 40 carbon atoms; and
(ii) up to 30% by weight—based on the total weight of the preparation—of a lipid phase comprising
(a) one or more lipids from the group of silicone oils and silicone waxes and
(b) one or more lipids from the group of nonpolar lipids with a polarity of ≧30 mN/m,
wherein the ratio of (a) to (b) is selected from the range of 1:3 to 3:1, with
(b) at least one filler material gas or propellant selected from the group consisting of linear and branched-chain, halogenated or nonhalogenated hydrocarbons, carbon dioxide, oxygen, compressed air, helium, krypton, xenon, radon, argon, nitrogen and dimethyl ether,
by releasing (a) and (b) from a pressurized container.

30. The method for preparing foam as claimed in claim 29, wherein the lipid phase is comprised of up to 40% by weight—based on the total weight of the lipid phase—lipids with a polarity of up to 30 mN/m.

31. The method for preparing foam as claimed in claim 29, wherein the preparation further comprises at least one hydrophilic emulsifier.

32. The method for preparing foam as claimed in claim 29, wherein the preparation further comprises at least one UV filter substance.

33. The method for preparing foam, as claimed in claim 29, wherein the at least one filler material gas or propellant includes carbon dioxide.
